SEQUENCE LISTING (6 sequences)

SEQ ID NO: 1 - DNA, 17 bp, Artificial Sequence
  Description of Artificial Sequence Synthetic primer
atggacaggcgccctga

SEQ ID NO: 2 - DNA, 20 bp, Artificial Sequence
  Description of Artificial Sequence Synthetic primer
ttgatgggcagcttggtttc

SEQ ID NO: 3 - DNA, 17 bp, Artificial Sequence
  Description of Artificial Sequence Synthetic probe
cacgccatgcgctgcgg

SEQ ID NO: 4 - DNA, 21 bp, Artificial Sequence
  Description of Artificial Sequence Synthetic primer
cgttggctacccgtgatattg

SEQ ID NO: 5 - DNA, 16 bp, Artificial Sequence
  Description of Artificial Sequence Synthetic primer
aatcgggagcggcgat

SEQ ID NO: 6 - DNA, 24 bp, Artificial Sequence
  Description of Artificial Sequence Synthetic probe
tgaccgcttcctcgtgctttacgg

(19) United States
(12) Patent Application Publication
(10) Pub. No.: US 2007/0224167 A1
(43) Pub. Date: Sep. 27, 2007

(54) Novel HCV inhibitor combinations and methods

(75) Inventors: Emillo Anthony Emini, Dresher, PA (US); Michael James Flint, Wayne, PA (US); Anita Yee Mei Howe, Paoli, PA (US); Bruce A. Malcolm, Paoli, PA (US); Stanley Mullen, Lincoln Park, NJ (US); Robert Orville Ralston II, Union, NJ (US); Xiao Tong, East Brunswick, NJ (US)

Correspondence Address: CHERYL H AGRIS PHD, PO BOX 806, PELHAM, NY, 10803, US

(73) Assignees: Schering Corporation, Kenilworth, NJ (US); ViroPharma Incorporated, Exton, PA (US); Wyeth, Madison, NJ (US)

(21) Appl. No.: 11/704,505

(22) Filed: Feb. 9, 2007

Related U.S. Application Data
(60) Provisional application No. 60/771,927, filed on Feb. 9, 2006.
(60) Provisional application No. 60/841,789, filed on Aug. 30, 2006.

(51) Int. Cl. A61K 38/21; A61K 31/7056; A61K 31/407; A61K 31/343

(52) U.S. Cl. 424/85.4; 514/412; 514/43; 514/469

(57) ABSTRACT

Novel hepatitis C virus (“HCV”) inhibitor combinations comprising an HCV protease inhibitor and HCV polymerase inhibitor, and optionally one or more biologically active agents, as well as uses of these combinations as HCV inhibitors and for treating hepatitis C and related disorders are disclosed.

### PRIORITY CLAIM

This application claims priority from application Ser. No. 60/771,927 filed Feb. 9, 2006 and application Ser. No. 60/841,789 filed Aug. 30, 2006, the contents of the latter of which is incorporated herein by reference.

### FIELD OF THE INVENTION

The invention is directed to novel hepatitis C virus (“HCV”) inhibitor combinations of an HCV protease inhibitor and HCV polymerase inhibitor as well as uses of these combinations as HCV inhibitors and for treating hepatitis C and related disorders. Furthermore, the invention is directed to a method for modulating HCV growth comprising administering an HCV protease inhibitor and HCV polymerase inhibitor. Kits and compositions containing these combinations are encompassed by the invention as well.

### BACKGROUND OF THE INVENTION

Identification or discussion of any reference in this section or any other section of this application shall not be construed as an admission that such reference is available as prior art to the present invention.

Hepatitis C virus (HCV) is a (+)-sense single-stranded RNA virus that is a member of the Flaviviridae family (reviewed in Purcell (1994) FEMS Rev. 14:181-192). HCV has been implicated as the major causative agent in non-A, non-B hepatitis (NANBH), particularly in blood-associated NANBH (BB-NANBH)(see, for example, WO 89/04669 and EP 381 216). HCV can lead to chronic hepatitis, cirrhosis of the liver, liver failure and hepatocellular carcinoma. It is one of the leading causes for liver transplantation.

Following infection by HCV, the viral RNA is translated into a polyprotein. This approximately 3,000-residue polyprotein is subsequently cleaved into individual proteins by host peptidases, as well as virally encoded proteases (see, e.g., U.S. Pat. No. 5,712,145). The HCV genome encodes structural proteins (required for virus assembly) and nonstructural proteins (required for replication). The structural proteins include a nucleocapsid protein (C) and envelope proteins (E1 and E2). The nonstructural proteins include: NS2, NS3, NS4A, NS4B, NS5A, and NS5B (reviewed in Bartenschlager (2000) J. General Virology 81:1631-1648). One of the proteins, NS3, is an approximately 68 kD protein, encoded by approximately 1893 nucleotides of the HCV genome, and has two distinct domains: (a) a serine protease domain consisting of approximately 181 of the N-terminal amino acids; and (b) an ATP-dependent RNA helicase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. The 6 kD NS4a protein is a cofactor for the serine protease activity of NS3. Another nonstructural protein, NS5B, is an RNA-dependent RNA polymerase that is essential for viral replication.

Current treatments for HCV include interferon and interferon in combination with ribavirin (see, e.g., Berenguer et al. (1998) Proc. Assoc. Am. Physicians 110(2): 98-112). A sustained clinical improvement is seen in approximately 50% of patients. Thus, the effectiveness of therapy for chronic hepatitis C is low. Moreover, therapy is often associated with considerable side effects. These therapies suffer from a low sustained response rate and frequent side effects. (See, e.g., Hoofnagle et al. (1997) N. Engl. J. Med. 336:347). No vaccine is currently available for HCV infection.

A number of HCV protease inhibitors have been disclosed. These include antioxidants (see, International Patent Application Publication No. WO 98/14181), inhibitors based on the 70-amino acid polypeptide eglin c (Martin et al. (1998) Biochem. 37:11459-11468), inhibitors affinity selected from human pancreatic secretory trypsin inhibitor (hPST1-C3) and minibody repertoires (MBip) (Dimasi et al. (1997) J. Virol. 71:7461-7469), cVₙHE2 (a “camelized” variable domain antibody fragment) (Martin et al. (1997) Protein Eng. 10:607-614), and .alpha.1-antichymotrypsin (ACT) (Elzouki et al.) (1997) J. Hepat. 27:42-28). Additionally, a ribozyme designed to selectively destroy hepatitis C virus RNA has recently been disclosed (see, BioWorld Today 9(217):4 (Nov. 10, 1998)).

Additionally, a number of peptide analogs have been disclosed that have been found to act as protease inhibitors (particularly, HCV NS3 protease inhibitors). See, for example, WO98/17679, Landro et al. (1997) Biochem. 36:9340-9348, Ingallinella et al. (1998) Biochem. 37:8906-8914, Llinas-Brunet et al. (1998) Bioorg. Med. Chem. Lett. 8:1713-1718, WO 98/17679, WO 98/22496, WO 99/07734, Marchetti et al. (1999) Syn Let S1:1000-1002, WO 00/09558, WO 00/09543, Tong et al., 2006, Antiviral Res. 70:28-38 and U.S. Pat. No. 7,012,066.

RNA polymerase inhibitors have also been disclosed. WO 2004/041201 discloses benzofuran compounds that can act as HCV RNA polymerase inhibitors.

However, a patient may become resistant to a particular treatment modality. There have been disclosures of HCV variants with reduced susceptibility to anti-HCV agents (see, for example, Krieger et al., 2001, J. Virol. 75: 4614-4624, and Lin et al., US Patent Pub. No. 2005/0136400. Thus, there is a need for new treatments and therapies for HCV infection. An object of this invention is to provide combinations useful in the treatment or prevention or amelioration of one or more symptoms of HCV. It is a further object herein to provide methods of treatment or prevention or amelioration of one or more symptoms of HCV.

### SUMMARY OF THE INVENTION

The invention is directed to a combination or combinations of (a) an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and (b) an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing. In the combination of the present invention, the above-mentioned HCV RNA polymerase inhibitor and above-mentioned HCV protease inhibitor or their isomeric forms or salts may be formulated into separate dosage forms or alternatively into a composition comprising said HCV RNA polymerase inhibitor and HCV protease inhibitor. In a particular embodiment, the invention is directed to a pharmaceutical composition comprising said HCV RNA polymerase inhibitor and said HCV protease inhibitor, which could, for example, be used to treat disorders associated with HCV and/or modulating the growth of HCV.

The invention is further directed to a method for modulating HCV RNA polymerase activity and/or HCV protease activity, particularly HCV serine protease activity in HCV infected cells in a subject in need thereof, comprising administering to said subject an amount of said HCV RNA polymerase inhibitor or a rotamer, tautomer or other isomeric form of said polymerase inhibitor or a pharmaceutically acceptable salt of any of the foregoing and said HCV protease inhibitor or an enantiomer, stereoisomer, rotamer, tautomer, racemate or other isomeric form of said protease inhibitor or a pharmaceutically acceptable salt of any of the foregoing in amounts effective to modulate said HCV RNA polymerase activity and/or HCV protease activity. In a particular embodiment, a composition comprising said HCV RNA polymerase inhibitor and said HCV serine protease inhibitor is administered. The subject is preferably a mammalian subject and most preferably a human subject.

The invention is further directed to a method for modulating HCV growth and/or activity in HCV infected cells in a subject in need thereof, comprising administering to said subject an amount of said HCV RNA polymerase inhibitor or a rotamer, tautomer or other isomeric form of said polymerase inhibitor or a pharmaceutically acceptable salt of any of the foregoing and said HCV protease inhibitor or an enantiomer, stereoisomer, rotamer, tautomer, racemate or other isomeric form of said protease inhibitor or a pharmaceutically acceptable salt of any of the foregoing or the composition of the present invention in amounts effective to modulate said HCV growth and/or activity. The cells would be mammalian cells and preferably human cells.

The invention is further directed to a method for modulating HCV RNA production and/or activity in HCV infected cells in a subject in need thereof, comprising administering to said subject an amount of said HCV RNA polymerase inhibitor or a rotamer, tautomer or other isomeric form of said polymerase inhibitor or a pharmaceutically acceptable salt of any of the foregoing and said HCV protease inhibitor or an enantiomer, stereoisomer, rotamer, tautomer, racemate or other isomeric form of said protease inhibitor or a pharmaceutically acceptable salt of any of the foregoing or the composition of the present invention in amounts effective to modulate said HCV growth and/or activity. In a particular embodiment, the rate of HCV RNA production is modulated. The cells would be mammalian cells and preferably human cells.

The invention is further directed to a method for treating a disorder associated with HCV comprising administering to a subject in need thereof an amount of said HCV RNA polymerase inhibitor or a rotamer, tautomer or other isomeric form of said polymerase inhibitor or a pharmaceutically acceptable salt of any of the foregoing and said HCV protease inhibitor or an enantiomer, stereoisomer, rotamer, tautomer, racemate or other isomeric form of said protease inhibitor or a pharmaceutically acceptable salt of any of the foregoing, or said composition of the present invention in amounts effective to treat said disorder.

The invention is further directed to a kit comprising the combination of the present invention, the above-mentioned HCV RNA polymerase inhibitor or a rotamer, tautomer or other isomeric form of said polymerase inhibitor or a pharmaceutically acceptable salt of any of the foregoing and HCV serine protease inhibitor or an enantiomer, stereoisomer, rotamer, tautomer, racemate or other isomeric form of said protease inhibitor or a pharmaceutically acceptable salt of any of the foregoing, as well as instructions for administering this combination.

The invention is also directed to the use of said HCV polymerase inhibitor and said HCV protease inhibitor in the manufacture of a medicament comprising said HCV polymerase inhibitor or a rotamer, tautomer or other isomeric form of said polymerase inhibitor or a pharmaceutically acceptable salt of any of the foregoing and said HCV protease inhibitor or an enantiomer, stereoisomer, rotamer, tautomer, racemate or other isomeric form of said protease inhibitor or a pharmaceutically acceptable salt of any of the foregoing in the same or different preparations for the treatment of disorders associated with HCV.

The invention further relates to a method for decreasing the emergence or the rate or frequency of the emergence of resistance to said HCV polymerase inhibitor or said HCV protease inhibitor in HCV infected cells in a subject comprising administering to said subject in need thereof an amount of the combination of the present invention effective to decrease the emergence of said resistance.

Other aspects of the invention will be apparent to those skilled in the art from the description contained herein and from the appended claims and figures.

### BRIEF DESCRIPTION OF THE FIGURES

FIG. 1 shows the plate set up for the 3-day replicon combination assay.

FIGS. 2A and 2B show the results of two experiments showing titration of SCH 503034 in the presence of increasing levels of HCV-796.

FIG. 3 shows the results of studies assaying the activity of HCV-796 on SCH 503034 resistant replicon cell lines.

FIG. 4 shows the activity of SCH 503034 in HCV 1b BB7 replicon (FIG. 4A) and in HCV 1a H77 replicon (FIG. 4B).

FIG. 5 shows the activity of HCV-796 in HCV 1b BB7 replicon (FIG. 5A) and in HCV 1a H77 replicon (FIG. 5B).

FIG. 6 shows the results of studies assaying the activity of SCH 503034 on HCV-796 resistant replicon cell lines.

FIG. 7 shows experimental design for the Three (3) Day Combination Assay. Each combination is conducted in four replicates in two independent experiments.

FIGS. 8A and 8B show the results of two experiments of the three day assay testing the combination of HCV-796 and SCH 503034 in the form of a Synergy Plot (95% confidence).

FIG. 9 shows the experimental design for the two-week combination assay.

FIG. 10 shows the impact of the combination therapy on HCV RNA levels over time (15 days). FIG. 10A shows the effect of 40 nM HCV-796 and 400 nM SCH 50304; FIG. 10B shows the effect of 40 nM HCV-796 and 800 nM SCH 50304; FIG. 10C shows the effect of 80 nM HCV-796 and 400 nM SCH 50304 and FIG. 10AD shows the effect of 80 nM HCV-796 and 800 nM SCH 50304. FIG. 10E is a summary graph showing all of the data collected.

FIG. 11 shows an analysis of the antiviral effect of the combination therapy using the Perelson bi-exponential model: dV/dt=p(1−ε)I−cV and dI/dt=β(1−η)Vμ−δI.

FIG. 12 shows the effect of the combination therapy on host cell GAPDH mRNA levels.

FIG. 13 shows a comparison of dose responses to SCH 503034 and HCV 796 on day 3 in short and long term replicon assays

FIG. 14 shows the effect of the combination therapy on the frequency of colony formation.

FIG. 15 shows the frequency of emergence of resistant colonies in a long term replicon assay in cells treated with the HCV-796 and SCH-503034 combination (combined results from three experiments).

FIG. 16 shows resistant colonies per duplicate well (results from two experiments) in cells treated with HCV-796 and SCH-503034 in combination. The number of colonies in each duplicate shown; TNTC: >800 colonies; NA: not available

FIG. 17 shows results from a 2-week combination assay (two experiments).

FIG. 18 shows replicon RNA reduction after 14-day combination treatment. For HCV-796, IC90=30 nM, for SCH 50304, IC90=400 nM. The Taqman detection limit is 3-4 log reduction.

FIG. 19 shows results from studies of replicon RNA reduction after an 1-day combination treatment. For HCV-796, IC90=30 nM, for SCH 50304, IC90=400 nM. The Taqman detection limit is 4-5 log reduction.

### DETAILED DESCRIPTION OF THE INVENTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The term “modulate” is used to mean alter the amount or rate of, for example, HCV RNA polymerase activity, HCV protease activity and/or HCV growth.

The term “treatment” means any process or method which ameliorates, inhibits or reverses one or more of the deleterious effects of HCV or which inhibits or slows the progress of HCV replication.

The term “combination” as used herein means the use of an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide, an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide and optionally one or more, or two or more other biologically active agents in separate or combined dosage forms, as well as a composition comprising said HCV RNA polymerase inhibitor, HCV protease inhibitor and optionally, one or more, or two or more biologically active agents.

HCV RNA Polymerase Inhibitor

The HCV RNA polymerase inhibitor used in the methods and combinations of the present invention may be a benzofuran. In a particular embodiment, the HCV RNA polymerase inhibitor is 5-cyclopropyl-2-(4-fluoro-phenyl)-6-[(2-hydroxy-ethyl)-methanesulfonyl-amino]-benzofuran-3-carboxylic acid methylamide disclosed in WO 2004/041201 (see Example 43, specifically incorporated herein by reference) and referred to in the Examples as HCV-796. This inhibitor can be obtained using methods known in the art as well as, for example, methods disclosed in WO 2004/041201. The HCV RNA polymerase inhibitor of the invention can form one or more pharmaceutically acceptable salts with inorganic and organic acids such as hydrochloric, sulfuric, acetic, lactic, or the like and with inorganic or organic bases such as sodium or potassium hydroxide, piperidine, ammonium hydroxide, or the like. The invention also includes tautomers, rotamers, and other isomeric forms of the HCV RNA polymerase inhibitor of the present invention. Therefore, the HCV RNA polymerase inhibitor used in the combinations, compositions, methods and kits of the present invention may exist in suitable isomeric forms.

HCV Protease Inhibitor

The HCV protease inhibitor used in the methods and combinations of the present invention may have the structure disclosed in U.S. Pat. No. 7,012,066 and in a particular embodiment is (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide and is referred to in the Examples as SCH 503034. The protease inhibitor of the combination of the present invention may be prepared using methods known in the art and, in particular, U.S. Pat. No. 7,012,066, (see Example 24, specifically incorporated herein by reference). As with the HCV RNA polymerase inhibitor, the HCV NS3/NS4A serine protease inhibitor of the present invention may form one or more pharmaceutically acceptable salts with organic or inorganic acids, or organic or inorganic bases. Examples of suitable acids for such salt formation include but are not limited to hydrochloric, sulfuric, phosphoric, acetic, citric, malonic, salicylic, malic, fumaric, succinic, ascorbic, maleic, methanesulfonic and other mineral and carboxylic acids well known to those skilled in the art. For formation of salts with bases, suitable bases are, for example, NaOH, KOH, NH₄OH, tetraalkylammonium hydroxide, and the like. The invention also includes tautomers, rotamers, enantiomers and other isomeric forms of the compound of the present invention. Therefore, the HCV serine protease inhibitor used in the combinations, compositions, methods and kits of the present invention may exist in suitable isomeric forms.

Uses

The combinations, methods, kits and compositions of the present invention may be used to both modulate HCV RNA polymerase activity and/or HCV protease activity and/or particularly, HCV growth in HCV infected cells and particularly HCV RNA production and even more particularly, the rate of emergence of resistant variants to one or more of the components of the combinations in HCV infected cells, particularly, in a subject in need thereof, such as a mammal and, in particular, a human. In a particular embodiment, the combinations, methods, kits and compositions of the present invention, may be advantageous in cells which have developed resistance to an HCV RNA polymerase inhibitor or HCV protease inhibitor. The combination, methods, kits and compositions of the present invention may also be used to treat HCV related disorders and/or infections caused by HCV.

The method of the present invention may also include in addition to administering the HCV RNA polymerase inhibitor and HCV protease inhibitor of the combination of the present invention, administering other biologically active agents including, but not limited to, one or more protease inhibitors, RNA polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and/or anti-infective compounds.

In a specific embodiment, the other biologically active agent includes, but is not limited to, Ribavirin (from Schering-Plough Corporation, Madison, N.J.) and Levovirin™ (from ICN Pharmaceuticals, Costa Mesa, Calif.), VP50406™ (from Viropharma, Incorporated, Exton, Pa.), ISIS 14803™ (from ISIS Pharmaceuticals, Carlsbad, Calif.), Heptazyme™ (from Ribozyme Pharmaceuticals, Boulder, Colo.), VX 497™ (from Vertex Pharmaceuticals, Cambridge, Mass.), Thymosin™ (from SciClone Pharmaceuticals, San Mateo, Calif.), Zadaxin™, Maxamine™ (Maxim Pharmaceuticals, San Diego, Calif.), mycophenolate mofetil (from Hoffman-LaRoche, Nutley, N.J.), ANA975™ (Anadys, San Diego, Calif.), Hiltonol™ (Oncovir Inc., Washington, D.C.), interferon (such as, for example, interferon-alpha, PEG-interferon alpha conjugates), interferon-alpha-n3 (from Hemispherx Biopharma), interferon-alpha-2b (from Biogen Idec), interferon-alpha-2b+ribavirin (Rebetron™ from Biogen Idec, Valeant Pharmaceuticals International), and the like. “PEG-interferon alpha conjugates” are interferon alpha molecules covalently attached to a PEG molecule. Illustrative PEG-interferon alpha conjugates include, but are not limited to, interferon alpha-2a (Roferon™, from Hoffman La-Roche, Nutley, N.J.) in the form of pegylated interferon alpha-2a (e.g., as sold under the trade name Pegasys™), interferon alpha-2b (Intron™ from Schering-Plough Corporation) in the form of pegylated interferon alpha-2b (e.g., as sold under the trade name PEG-Intron™), interferon alpha-2c (Berofor Alpha™, from Boehringer Ingelheim, Ingelheim, Germany), consensus interferon as defined by determination of a consensus sequence of naturally occurring interferon alphas (Infergen™, Advaferon™, Infarex™, from Amgen, Thousand Oaks, Calif.), as well as interferon-beta and interferon-gamma, CpG 10101 (from Coley Pharmaceutical Group). Other biologically active agents include, but are not limited to, Tarvacin™ (from Peregrine Pharmaceuticals, USA), R 7025 (from Maxygen, USA), EHC 18 (from Enzo Biochem (Israel) and Enzo Biochem (USA)), Thymalfasin (from University of Texas at Austin, USA), NOV 205 (from BAM Russia), Ursodeoxycholic acid (from Alfa-Schiapparelli-Wasserman Group, Sanofi-Aventis), Civacir™ (from Nabi Biopharmaceuticals USA), XTL 6865 (from XTL Biopharmaceuticals, Israel), BLX 833 controlled-release (Locteron™ from Biolex, OctoPlus), Albuferon (from HGS/Novartis), Omega IFN (from Intarcia Therapeutics), Multiferon (from Viragen), INNO 101 vaccine (Innogenetics), IC 41 vaccine (from Intercell, Austria), HCV E1/E2 vaccine (from Chiron Corporation/St. Louis University), HCV ISCOM vaccine (from Chiron Corporation/CSL Limited), GI 5005 vaccine (from Globe Immune), GNS 037, a viral entry inhibitor (from Genoscience, France), HRC203, a ribavirin-hemoglobin conjugate (from Hemosol Corp., Canada), Taribavirin (from Valeant Pharmaceuticals International, USA), Viramidine (from Valeant Pharma), Suvus (from Bioenvision), HCV I.E.T. (from Transition Therapeutics), R7128 (from Roche/Pharmasset), AVI-4065 antisense (from AVI Biopharma), Celgosivir, a replication inhibitor (from MIGENIX), and BIVN 401, a replication inhibitor (from Oklahoma Medical Research Foundation).

Other biological agents include but are not limited to one or more of the following protease/polymerase inhibitors: VX 950™ (from Vertex Pharmaceuticals, Cambridge, Mass.), GS-9132 (from Gilead, Foster City, Calif.), ITMN-B™ (from Intermune, Brisbane, Calif.), ITMN-191 (from Intermune, Brisbane, Calif.), Valopicitabine (NM283) (from Idenix, Cambridge, Mass.), RO-4048™ (from Pharmassett, Princeton, N.J.), A-782759™ (from Abbott Laboratories, Abbott Park, Ill.), XTL-2125™ (from XTL Biopharmaceuticals, New York, N.Y.), MK 0608 (from Merck & Co (USA)), A-689 (from Arrow Therapeutics, United Kingdom), A-831 (from Arrow Therapeutics, United Kingdom), R 7128 (from Pharmasset, USA), R-1479 (from Argenta Discovery, Roche), 2′-deoxy-2′-fluorocytidine, FdC (from Emory University, Pharmasset), JTK 003 (from Japan Tobacco, Japan), R 1626 (from Novartis), PSI-6130 (from Pharmasset), TJ 9 (from Janssen Pharmaceutical KK), Telaprevir (from Vertex Pharmaceuticals International (USA)), LB 84451 (from LG Life Sciences, South Korea), MW 559 (from Merck Sharp & Dohme-Sigma-Tau (JV)), ITMN 191 (from Array BioPharma, InterMune), GW 0014 (from GlaxoSmithKline, United Kingdom), GAPC 6336 (from Applera Corporation, Bristol-Myers Squibb), IFN-beta-1a (Rebif from Ares Serono).

The HCV RNA polymerase inhibitor and HCV protease inhibitor described herein and used in the method of the present invention along with optionally one or more other biological agents can be administered concurrently. The treatment with both compounds can be in the same daily dose or in separate doses. Concurrent administration of the HCV RNA polymerase inhibitor and HCV protease inhibitor means that effective concentrations of both inhibitors are simultaneously present in the patient.

Alternatively, the HCV RNA polymerase inhibitor and HCV protease inhibitor described herein and used in the method of the present invention along with optionally one or more other biological agents can be administered sequentially. The sequential therapy can be within a reasonable time after the completion of the first therapy before beginning the second therapy. In yet another embodiment, the HCV RNA polymerase inhibitor and HCV protease inhibitor may be administered concurrently followed by or following administration of other biological agents. Other biological agents may be administered separately or in combination with the HCV inhibitor, HCV protease inhibitor and/or one or more other biological agents set forth above.

In yet another embodiment, in addition to sequential and concurrent administration of the combination of the present invention, intermittent administration of the therapeutic combination regimen may also be done in order to minimize side effects while retaining or improving antiviral response (see, Martinez-Picado, J. et al., 2003, Ann. Intern. Med. 139:81-89). For example, a patient could receive the combination (either sequentially or concurrently) for a period of time and then the patient could discontinue the combination for a time or the patient could receive a drug regimen other than the combination of the present invention. The alternating of the combination of the present invention and the alternative drug regimen can be repeated one or more times according to the individual's need and the professional judgment of the person administering or supervising the administration of the combination therapy.

The dosages for both concurrent and sequential combination therapy will depend on absorption, distribution, metabolism, and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy.

In a particular embodiment, the compounds used in the method of the present invention may be administered orally, rectally, parenterally, such as by intramuscular injection, subcutaneous injection, intravenous infusion or the like, intracisternally, intravaginally, intraperitoneally, locally, such as by powders, ointments, or drops, or the like, or by inhalation, such as by aerosol or the like, taking into account the nature and severity of the infection being treated. Depending on the route of administration, the HCV RNA polymerase inhibitor is preferably administered at dosage levels of about 25 to 3000 mg per day (e.g., 25 mg, 50 mg, 100 mg, 150 mg, 200 mg, 250 mg, 300 mg, 350 mg, 400 mg, 450 mg, 500 mg, 750 mg, 1000 mg, 1050 mg, 2000 mg, 3000 mg per day). In one preferred embodiment, the HCV RNA polymerase inhibitor is administered at a dosage range of about 100 mg to about 3000 mg per day. The dosage of HCV RNA polymerase inhibitor may be administered as a single dose (i.e. QD) or divided over 2-4 doses (i.e., BID, TID or QID) per day. The HCV RNA polymerase inhibitor used in the method of the present invention may be administered from 1 to 4 times a day. The HCV protease inhibitor is preferably administered at a dosage range of about 100 to about 3600 mg per day (e.g., 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg per day). In one preferred embodiment, the HCV protease inhibitor is administered at a dosage range of about 400 mg to about 2500 mg per day. The dosage of HCV protease inhibitor may be administered as a single dose (i.e., QD) or divided over 2-4 doses (i.e., BID, TID, or QID) per day. Preferably, the HCV protease inhibitor is administered orally. Other biologically active agents may be administered at a dosage range of about 1.0 to about 1000 mg/kg of subject body weight per day, more preferably 0.1 to about 100 mg/kg of subject body weight per day, one or more times a day, to obtain the desired therapeutic effect. The actual dosages of the HCV RNA polymerase inhibitor and HCV protease inhibitor and other biologically active agent(s) employed in the present invention may be varied depending upon the patient's age, sex, weight and severity of the condition being treated and other factors. Methods for calculating an appropriate dosage for a given patient are well known to those skilled in the art.

Compositions

In another embodiment, this invention provides compositions, in particular, pharmaceutical compositions comprising the one or more compounds used in the method of the present invention as an active ingredient. The pharmaceutical compositions generally additionally comprise a pharmaceutically acceptable carrier diluent, excipient or carrier (collectively referred to herein as carrier materials). The carrier materials are suitably selected with respect to the intended form of administration, and include, but are not limited to, oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Suitable binders include, but are not limited to, starch, gelatin, natural sugars, corn sweeteners, natural and synthetic gums such as acacia, sodium alginate, carboxymethylcellulose, polyethylene glycol and waxes. Lubricants that may be mentioned for use in these dosage forms include, but are not limited to, boric acid, sodium benzoate, sodium acetate, sodium chloride, and the like. Disintegrants include starch, methylcellulose, guar gum and the like. Sweetening and flavoring agents and preservatives may also be included where appropriate.

Additionally, the compositions of the present invention may be formulated in sustained release form to provide the rate of controlled release of any one or more of the components or active ingredients to optimize the therapeutic effects, i.e. HCV inhibitory activity and the like. Suitable dosage forms for sustained release include, but are not limited to, layered tablets containing layers of varying disintegration rates or controlled release polymeric matrices impregnated with one or more active components and shaped in tablet form or capsules containing such impregnated or encapsulated porous polymeric matrices.

Liquid form preparations suitable in the practice of the invention include solutions, dispersions, suspensions and emulsions. As an example, liquid form preparations may have water or water-propylene glycol solutions for parenteral injections or sweeteners and/or pacifiers for oral solutions, suspensions and emulsions. Liquid form preparations may also include solutions for intranasal administration.

Aerosol preparations suitable for inhalation may include without limitation liquid preparations or solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also suitable in the practice of this invention are solid form preparations that are intended to be converted, shortly before use, to liquid form preparations for either oral or parenteral administration. Such liquid forms include solutions, suspensions and emulsions.

Preferably, the composition of the present invention is in a unit dosage form. In such form, the preparation is subdivided into suitably sized unit doses containing appropriate quantities of the active components, e.g., an effective amount to achieve the desired purpose.

Kits

The invention is further directed to kits containing the HCV RNA polymerase inhibitor or a rotamer, tautomer or other isomeric form of said polymerase inhibitor or a pharmaceutically acceptable salt of any of the foregoing and the HCV serine protease inhibitor or an enantiomer, stereoisomer, rotamer, tautomer, racemate or other isomeric form of said protease inhibitor or a pharmaceutically acceptable salt of any of the foregoing used in the combinations, compositions and methods of the present invention, as well as instructions for administration. The HCV RNA polymerase inhibitor and HCV serine protease inhibitor can be packaged separately or together. Furthermore, the kit may also comprise other biological agents.

### EXAMPLES

The Examples exemplified below describe results from studies indicating favorable cross-resistance profile of two HCV inhibitors and enhanced anti-replicon activity mediated by the combined use of both compounds.

The combined antiviral effect of an inhibitor of the HCV NS3/NS4a protease, (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide and hereinafter referred to in the Examples as SCH 503034, and a non-nucleoside inhibitor of the viral polymerase, 5-cyclopropyl-2-(4-fluorophenyl)-6-[(2-hydroxy-ethyl)-methanesulfonyl-amino]-benzofuran-3-carboxylic acid methylamide, and hereinafter referred to in the Examples as HCV-796, is evaluated using wild-type genotype 1b HCV replicon cells. Each compound is individually assessed for its ability to inhibit the activity of variant replicons exhibiting reduced susceptibility to other inhibitor.

As will be described in further detail below, the combination of SCH-503034 and HCV-796 notably enhanced replicon inhibition in treated cells, in a dose-dependent manner, compared with the effect of each inhibitor used alone. The antiviral effect of the combination was at least additive. No cytotoxicity was observed. SCH-503034 exhibited equivalent inhibitory activity against the wild-type replicon and replicon variants expressing one or more polymerase amino acid substitutions that engender reduced susceptibility to HCV-796. The inhibitory effect of HCV-796 against replicon variants with one or more protease amino acid substitutions mediating reduced susceptibility to the protease inhibitor was found to be identical to that observed against the wild-type replicon. The combination significantly reduced the frequency of emergence of resistant colonies compared to each inhibitor used alone.

The anti-replicon activity of the combination of SCH-503034 and HCV-796, as well as the activity of each compound against replicons with reduced susceptibility to the other compound, strongly support the combined use of these two inhibitors in patients with HCV. The cell-culture replicon data suggest that the in vivo antiviral effects of the combination will be notably improved over the effects seen to date with monotherapy. Importantly, compared with monotherapy, the combination will likely impose a greater genetic barrier to the selection of clinically resistant viral variants.

Comparison of Antiviral Response to the SCH 503034 and HCV-796

Inhibition of Replicon RNA Levels (3-Day Assay)

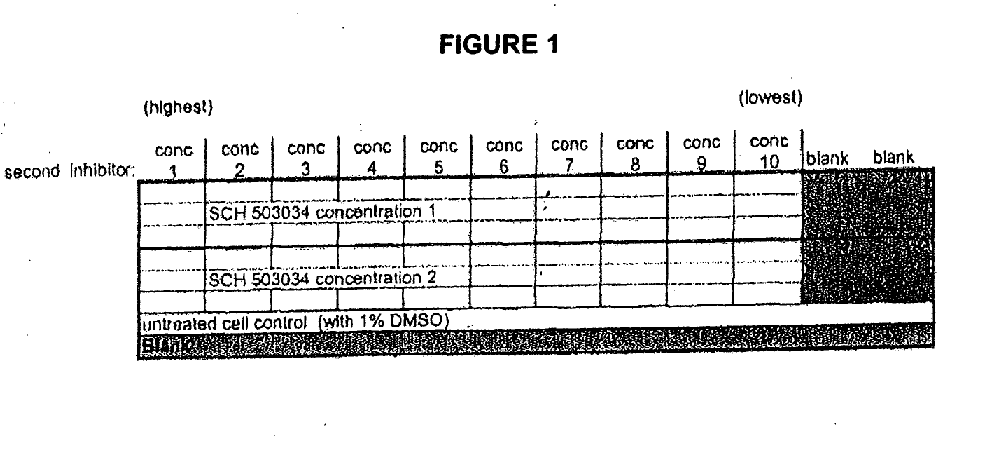

Replicon cells are seeded at ˜5000 cells/well in 96-well collagen I-coated Biocoat plates (Becton Dickinson). Twenty-four hrs post-seeding, inhibitors diluted in DMSO are added to replicon cells (Huh-7 cells). The final concentrations of DMSO and fetal bovine serum are 1% and 10%, respectively. SCH 503034 is serially diluted at 1:2 for a 10-point titration. To each concentration of the SCH 503034, the second inhibitor, HCV-796 or rhIFN-α2b control is titrated in. IFN-α is serially diluted at 1:3, whereas HCV RNA polymerase inhibitor, HCV-796, is serially diluted at 1:2. The final starting concentration is 2.5 μM for SCH 503034, 100 IU/ml for IFN-α (IntronA), and ˜5×IC₉₀ for HCV-796. All samples are tested in triplicate. A schematic of the plate set-up is shown in FIG. 1. Media and inhibitors are refreshed daily for 3 days at which point the cells are washed with PBS and lysed in 1× cell lysis buffer (Ambion cat #8721). The replicon RNA level is measured using real time PCR (Taqman assay) The amplicon is located in 5B. The PCR primers are: 5B.2F, ATGGACAGGCGCCCTGA (SEQ ID NO:1) and 5B.2R, TTGATGGGCAGCTTGGTTTC (SEQ ID NO:2). The probe sequence is FAM-labeled CACGCCATGCGCTGCGG (SEQ ID NO:3). GAPDH RNA is used as an endogenous control and is amplified in the same reaction as NS5B (multiplex PCR) using primers and VIC-labeled probe recommended by the manufacturer (PE Applied Biosystem). The real-time RT-PCR reactions are run on an AB1 PRISM 7900HT Sequence Detection System using the following program: 48° C. for 30 min, 95° C. for 10 min, 40 cycles of 95° C. for 15 sec, 6° C. for 1 min. The dCT values (CT_{5B}-CT_{GAPDH}) are plotted against SCH 503034 concentration and fitted to the sigmoid dose response model using SAS (SAS Institute Inc.) or Graphpad PRISM software (Graphpad Software Inc), IC₅₀ is defined as the drug dose necessary to achieve dCT=1 over the baseline. IC₉₀ is the drug dose necessary to achieve dCT=3.2 over the baseline.

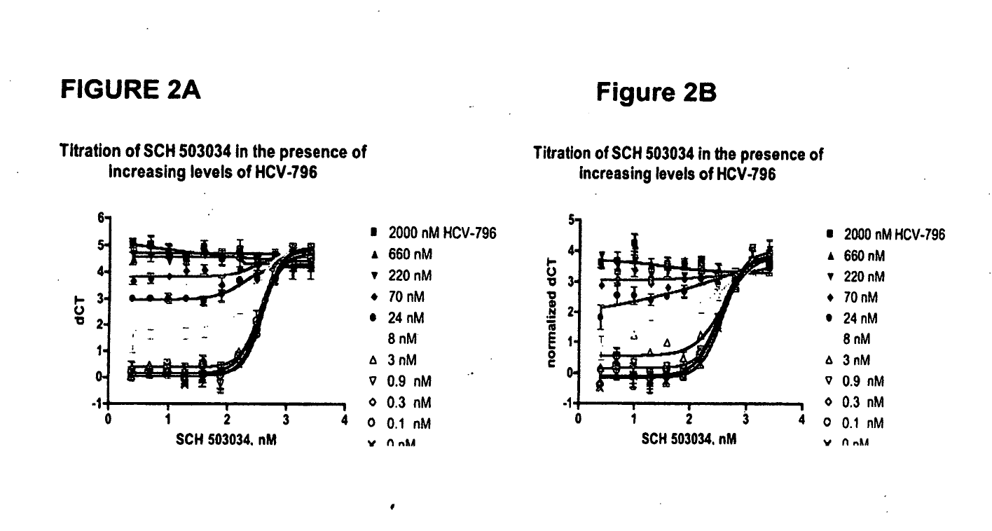

The results are shown in FIGS. 2A and 2B. An increase in dCT corresponds to a decrease in RNA level. It is evident that the combination of SCH 503034 and HCV-796 provides increased inhibition of HCV replicon RNA levels. Thus, the inhibitory activity of SCH 503034 and HCV-796 combination is at least additive.

HCV-796 Cross-Resistance Study Using Replicon Variants with Reduced Susceptibility

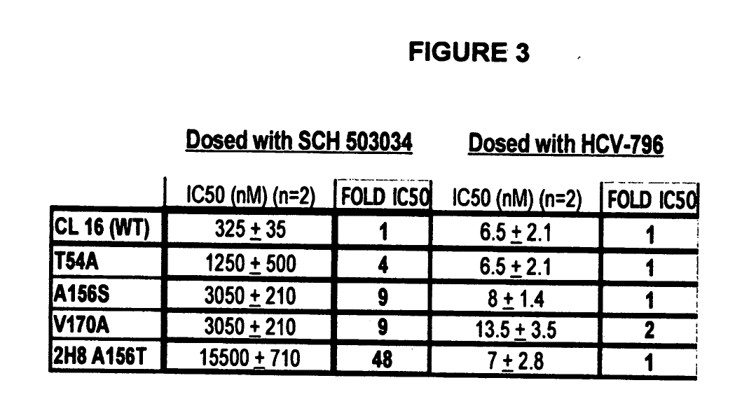

The 3-day replicon assay described above is carried out using wild type replicon and replicons containing the following resistance mutations in the HCV NS3 protease: T54A, A156S, and A156T, V170A. Replicons containing the mutations A156S, T54A and V170A are grown in a Huh7 cell line. A replicon containing the mutation A156T in the 2H8 subclone of the Huh7 cell line. The results are shown below in FIG. 3. The results indicate that HCV-796 is active on replicon cell lines containing SCH 503034 resistance mutations.

Evaluation of the Antiviral Activities of the HCV NS3/NS4a Protease Inhibitor and HCV RNA Polymerase Inhibitor

Material and Methods

Test and Control Articles

HCV genotype 1b, BB7 replicon-containing cell line is derived from a human hepatoma cell line (Huh7). A genotype 1a (H77 isolate; GenBank Accession #AF009606) is derived from replicon-containing cell line (Huh7-1a). The cell lines are cultured at 37° C. and 5% CO₂ in Dulbecco's Modified Eagle Media (D-MEM; Invitrogen #11965-084) containing 10% fetal bovine serum (FBS; HyClone #SH300070) supplemented with 1% penicillin/streptomycin (Invitrogen #15140-122), 1% non-essential amino acids (Invitrogen #11140-050), 0.66 mM HEPES buffer, pH 7.55 (Invitrogen #15630-080), and 1 mg/mL G418 (Geneticin®, Invitrogen #11811-031 or #10131-027). Genotype 1a and genotype 1b replicon-containing cell lines contain approximately 1000 and 2000 RNA genome equivalents per cell, respectively, when maintained in a subconfluent monolayer in the presence of 1 mg/mL G418. For compound testing, G418 is eliminated and the FBS concentration is reduced to 2%.

Quantification of HCV and 18S Ribosomal RNAs

At the end of the incubation period, the replicon-containing cells are lysed in 150 μL of lysis buffer provided in the RNeasy 96 Kit (Qiagen #74181). Total cellular RNA is extracted according to the manufacturer's protocol and eluted in 150 μL of nuclease-free water. TaqMan reactions are assembled in a 384-well plate according to the protocol provided in the TaqMan One Step RT-PCR Master Mix Reagents Kit (ABI #4309169) in a final volume of 20 μL. Included in the reaction mixture are 5 μL of RNA sample, 0.2 μM each of the forward primer (HCV[neo]: 5′-CGTTGGCTACCCGTGATATTG-3′ (SEQ ID NO:4)), reverse primer (HCV[neo]: 5′-AATCGGGAGCGGCGAT-3′ (SEQ ID NO:5)), and HCV probe (HCV[neo]: 5′-(6FAM)-TGACCGCTTCCTCGTGCTTTACGG-(TAMRA)-3′ (SEQ ID NO:6)). For duplexed RT-PCR quantifying both HCV RNA and 18S rRNA, 0.08 μM rRNA forward primer, 0.1 μM rRNA reverse primer, and 0.2 μM rRNA probe are added (ABI #4308329). The RT reaction is carried out at 48° C. for 30 min followed by a denaturation step at 95° C. for 10 min. The PCR amplification is conducted in 40 cycles; each cycle consisted of 95° C. for 15 sec followed by 60° C. for 1 min. Both steps are performed using the ABI Prism 7900HT Sequence Detection System (PE Biosystems).

The amounts of the HCV and 18S ribosomal RNAs in each sample are estimated by comparing the Ct cycles with those in the corresponding standard curves. HCV RNA used for the construction of the standard curve is prepared by extracting the total RNA from the Huh7-Clone A using the RNeasy maxi kit (Qiagen # 75162). The total RNA that is used for preparing the standard curve of the rRNA is quantified by O.D.₂₆₀ measurement. Compound dose response is measured in a 10-point, 3-fold serial dilution series performed in triplicates and subjected to the same corresponding RT-PCR conditions as described above. The concentration that inhibits 50% of the replicon RNA (EC₅₀) for each assay is calculated using the MDL LSW Data Analysis™ software in Microsoft Excel™. The amounts of HCV are expressed as HCV RNA copies and μg total RNA using rRNA as the surrogate marker for the quantification.

Combination Analysis

The combined antiviral effect of HCV-796 and SCH 503034 is monitored using a three-dimensional analytical method (MacSynergy™ II). This method examines drug combinations using the Bliss independence null model that is based on statistical probability and assumes that two drugs act independently to inhibit replication. Using this method, the theoretical additive interactions are calculated from the dose response curves of the individual drugs acting alone. The theoretical additive effects are then subtracted from the experimentally determined effects to reveal a difference in dose-response surface. The resulting surface appears as a horizontal plane at 0% difference if the interactions are additive. Any peaks above the plane are indicative of a greater than expected effect (synergy). Conversely, peaks appearing below the plane are indicative of a less than expected effect (antagonism). The confidence intervals around the experimental dose-response surface are used to evaluate the data statistically and the volume of the peaks is calculated to quantify the volume of synergy or antagonism produced. According to Prichard and Shipman (Prichard M N, Aseltine K R, Shipman J C. MacSynergy II. Version 1.0. User's manual: University of Michigan, Ann Arbor; 1993). a general guideline for the volume of synergism and antagonism is summarized as follows:

TABLE 1
Guideline for Synergism and Antagonism

| Volume of synergy or antagonism | Interpretation |
|---|---|
| +25 to −25 | Additive |
| +25 to +50 | Minor but significant synergistic |
| −25 to −50 | Minor but significant antagonistic |
| +50 to +100 | Moderately synergistic |
| −50 to −100 | Moderately antagonistic |
| >+100 | Strong synergistic |
| <−100 | Strong antagonistic |

Intracellular Antiviral Activities in HCV Replicon

Genotype 1b (BB7) and 1a (H77) cells are seeded in 96-well plates at a sub-confluent density (7000 cells/well) in medium containing 2% FBS without G418. HCV-796 and SCH 503034 solubilized with 100% dimethylsulfoxide (DMSO) are added to wells using a 10-point, 3-fold and 2-fold respectively serial dilution series, with a final DMSO concentration of 0.5% and a final volume of 200 μL. The final concentrations for HCV-796 are 0, 0.1, 0.4, 1.1, 3.3, 10.0, 30.0, 90.0, 270.0, 810.0 and 2,430 nM, and the final concentrations for SCH 503034 are 3.1, 6.3, 12.5, 25, 50, 100, 200, 400, 800, 1600 and 3200 nM. The plates are incubated for 72 hours at 37° C. and 5% CO₂. Under these conditions, the cells are approximately 25% confluent at the time of seeding and 80-90% confluent after 72 hours. At the end of the incubation period, total RNA is extracted from replicon containing cells using an RNeasy 96 Kit (Qiagen #74181) according to the manufacturer's protocol. The extracted RNA from each well is eluted in 150 μL of nuclease-free water. The amounts of HCV, rRNA and GAPDH RNAs are quantified using the TaqMan RT-PCR assay.

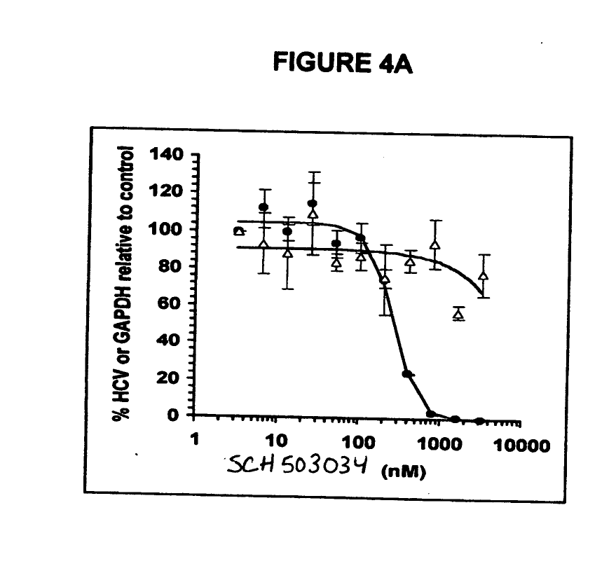

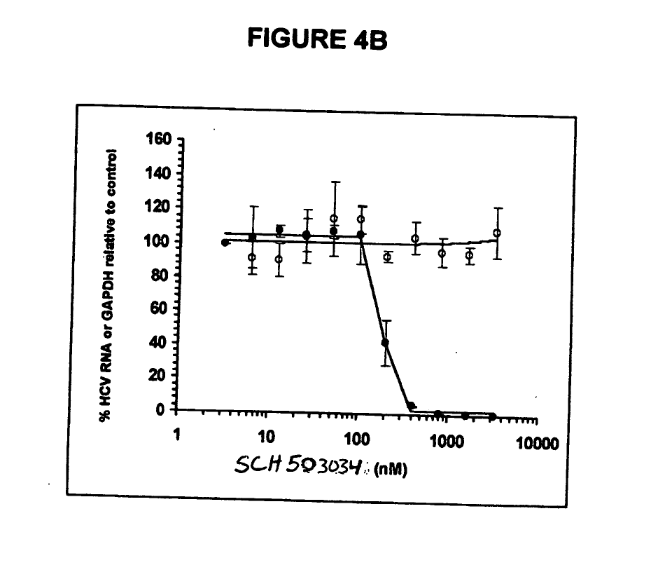

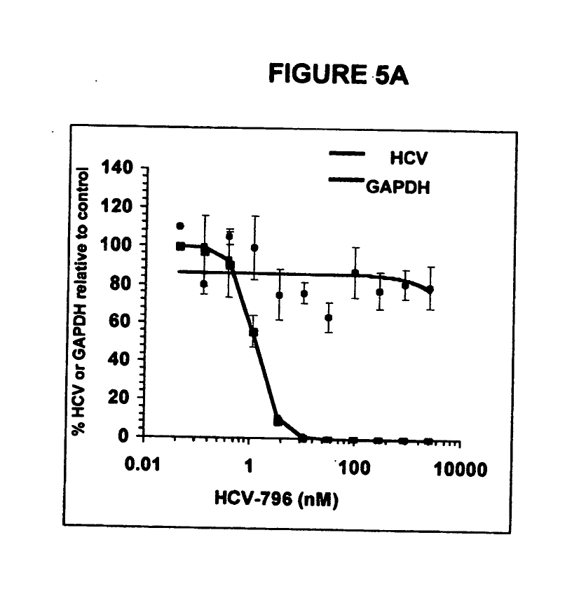

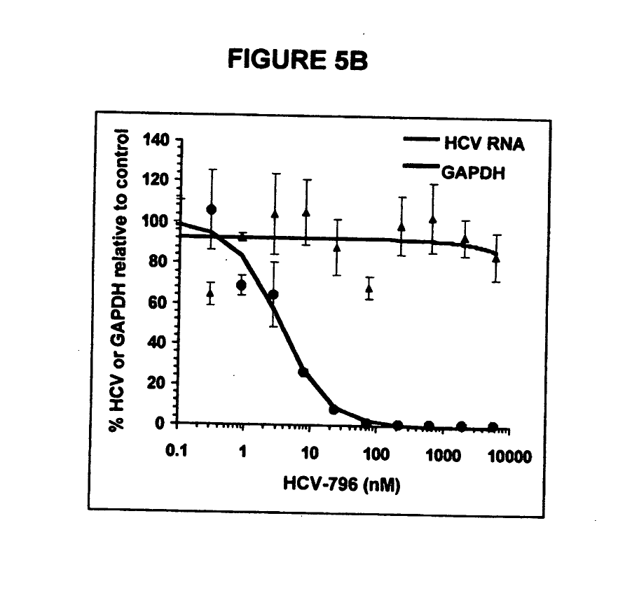

The results are shown in FIGS. 4 and 5. FIG. 4 shows the activity of the HCV protease inhibitor SCH 503034 where an HCV RNA EC₅₀=268±29 nM and GAPDH EC₅₀>3200 nM are obtained in FIG. 4A and an HCV RNA EC₅₀=188±18 nM and GAPDH EC₅₀>3200 nM are obtained in FIG. 4B. No difference in EC₅₀ is observed when changing media daily vs. single dose in 3 days. FIG. 5 shows the activity of the RNA polymerase inhibitor HCV-796 where an HCV RNA EC₅₀=1.1±0.2 nM and GAPDH EC₅₀>2430 nM is obtained in FIG. 5A and an HCV RNA EC₅₀=2.5±1.7 nM GAPDH EC₅₀>5600 nM is obtained in FIG. 5B.

Susceptibility of HCV-796 Resistant Replicons to SCH 503034

The antiviral activity of SCH 503034 against the replicon variants that have shown reduced susceptibility to HCV-796 is evaluated. Briefly, the replicon-containing cells are seeded in 96-well plates at a subconfluent density (7000 cells/well) in a medium containing 2% FBS without G418. SCH 503034 solubilized with 100% dimethylsulfoxide (DMSO) is prepared in a 10-point, 2-fold dilution series, with a final DMSO concentration of 0.5% and a final volume of 200 mL. The final concentrations for SCH 503034 are 3.1, 6.3, 12.5, 25, 50, 100, 200, 400, 800, 1600 and 3200 nM. The plate is incubated for 72 hours at 37° C. and 5% CO₂ before quantification of HCV and GAPDH RNAs.

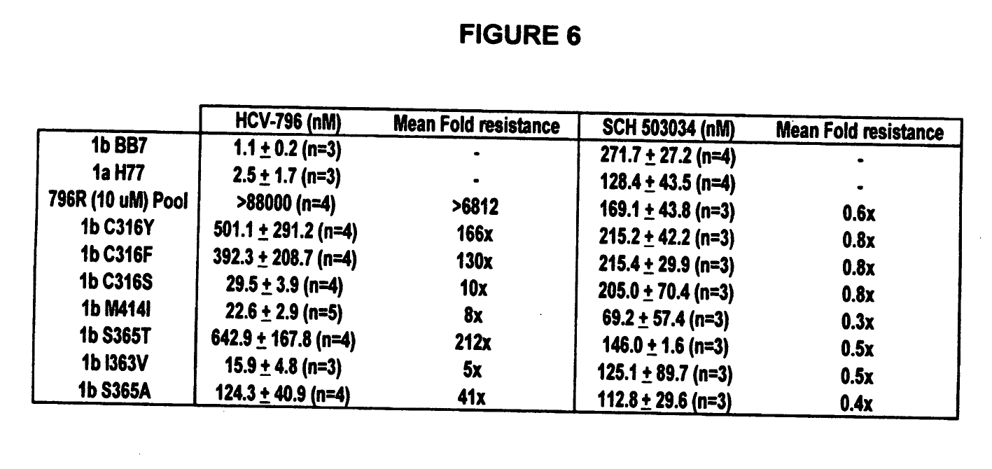

The results are shown in FIG. 6. The results indicate that SCH 503034 is active against replicons that have reduced susceptibility to HCV-796, including C316Y.

3-Day Combination Assay

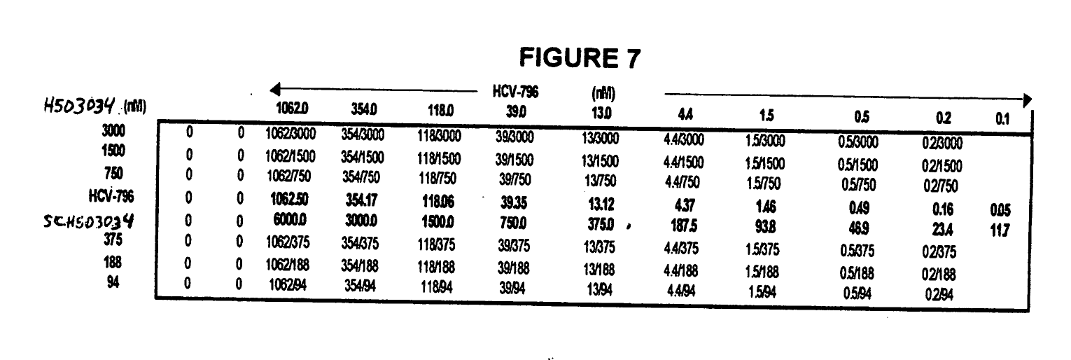

Huh7 cells containing the HCV genotype 1b (BB7) replicon are seeded at sub-confluent density (7000 cells per well in a 96-well plate) in a medium containing 2% FCS supplemented with 1% penicillin/streptomycin and 1% non-essential amino acids without G418. The cells are incubated at 37° C. in 5% CO₂ for 3-4 hours before compound addition. Under these conditions, cells are in an active growing state and reach confluence at the end of the 72-hour incubation with the compounds. The 10 mg/mL HCV-796 DMSO stock is diluted in 100% DMSO followed by stepwise 3-fold serial dilutions in culture medium. Fifty microliters of the diluted HCV-796 solution are added to the wells containing the cells. The final concentrations of HCV-796 are 0.1, 0.2, 0.5, 1.5, 4.4, 13, 39, 118, 354, 1062 nM. Similarly, the SCH 503034 stock is stepwise diluted in culture medium and added to the cells at final concentrations of 94, 188, 375, 750, 1500, 3000 nM. The dose responses for HCV-796 (1062-0.1 nM) and SCH 503034 (6000-12 nM) alone are run in parallel in each plate. All wells are adjusted to a final concentration of 0.5% DMSO. A total of 4 replicate plates with the layout described above are prepared. The cells are incubated with the compounds in 5% CO₂ at 37° C. for 72 hours before analysis for HCV and 18S ribosomal RNAs. The layout of combinations in the assay plate is shown in FIG. 7.

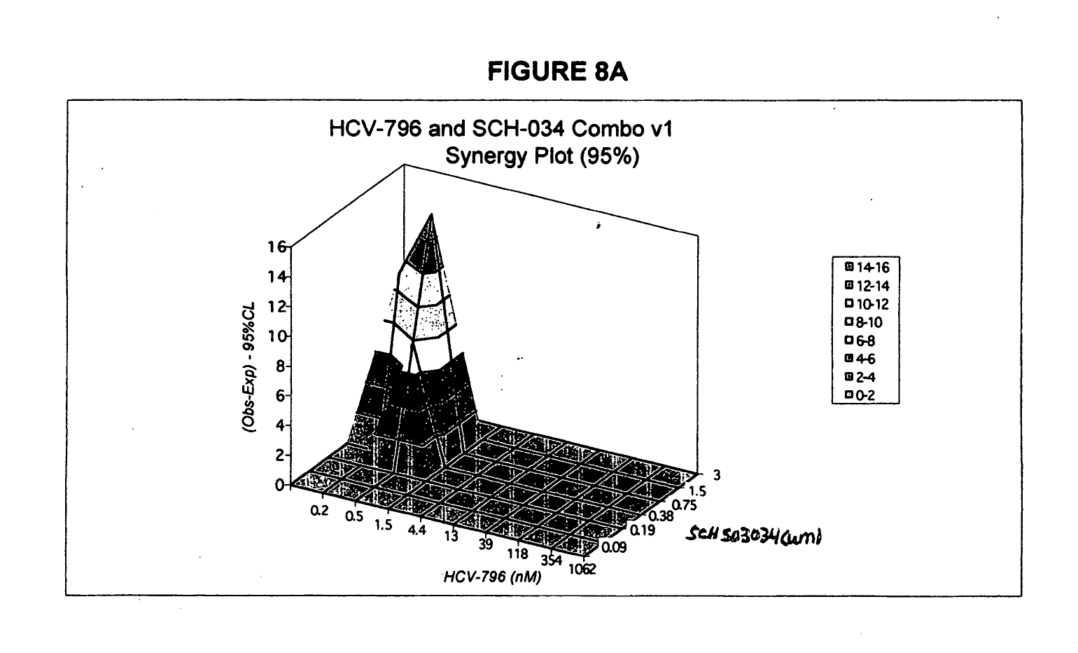

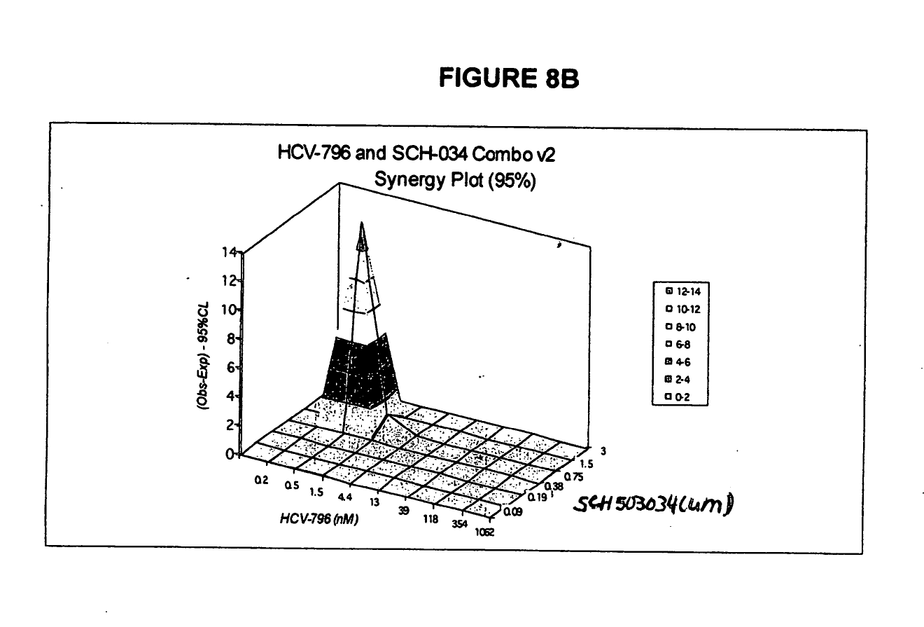

The results from two experiments are shown in FIGS. 8A and 8B. The combination of HCV-796 and SCH 503034 results in at least additive antiviral activity.

2-Week Combination Assay

Study A

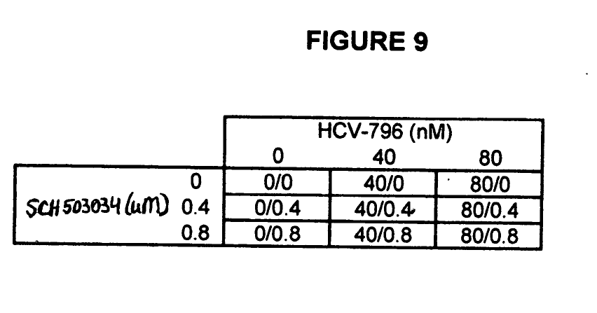

Huh7-BB7 cells are plated at a density of 2-3×10⁵ cells/T25 flask and cultured in DMEM medium with 2% FCS in the absence of G418. The cells are treated with various concentrations of HCV-796 and SCH 503034 as indicated in FIG. 9. DMSO concentration in both drug-treated and control cells is 0.5% (v/v). Tissue culture plates are incubated in a 37° C. incubator containing 5% CO₂. When cells reach about 80% confluence (about 2-3 days), cells are passaged in a 1:3 dilution and the old medium is replaced with fresh medium containing the compounds at the corresponding concentrations. As a control, Huh7-BB7 cells are passaged in parallel with the same medium except no compound is added. Cell pellets containing 2×10⁵ cells are collected every two to three days, lysed with 150 μL Qiagen lysis buffer provided in the RNasey 96 Kit (Qiagen #74181) and stored at −70° C. before analysis. Total RNA is extracted according to the manufacturer's protocol and eluted in 150 μl of nuclease-free water. The level of HCV RNA is quantified by quantitative Taqman RT-PCR as described above.

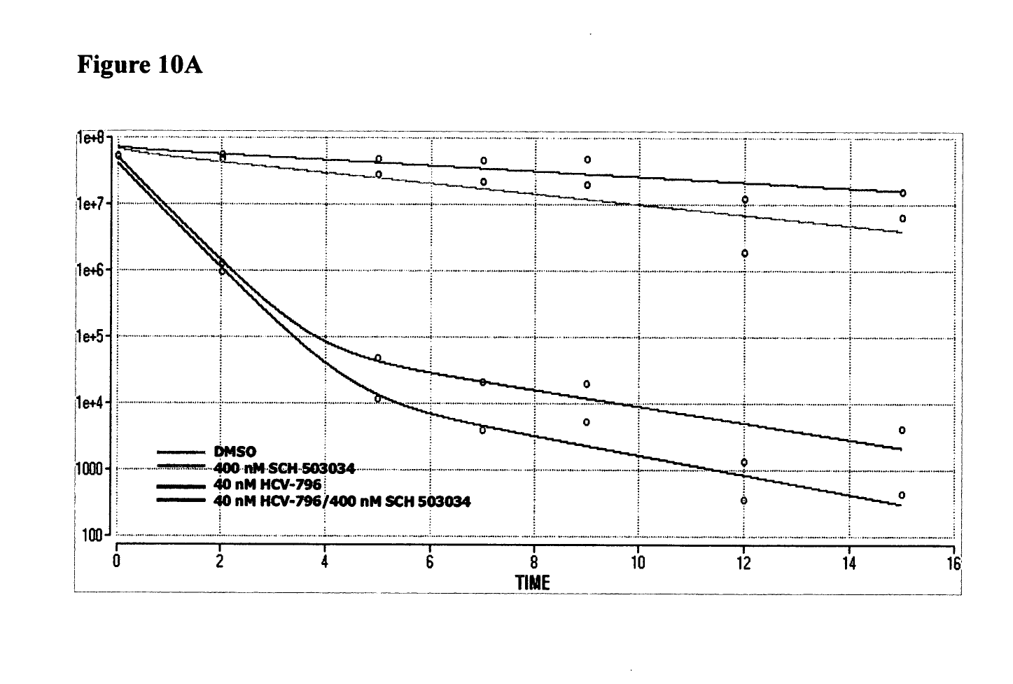

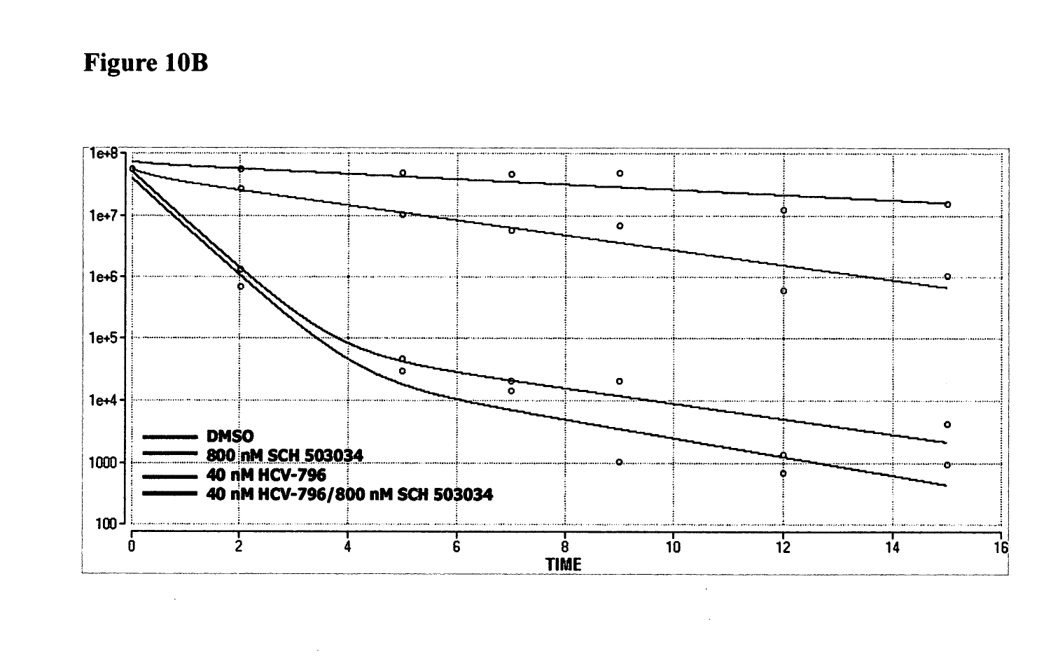

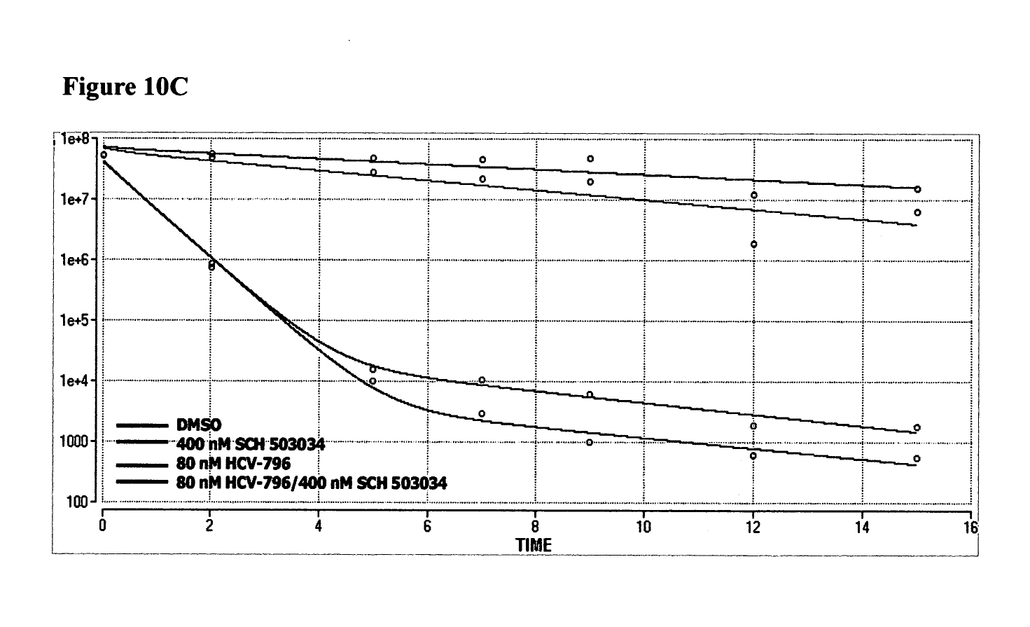

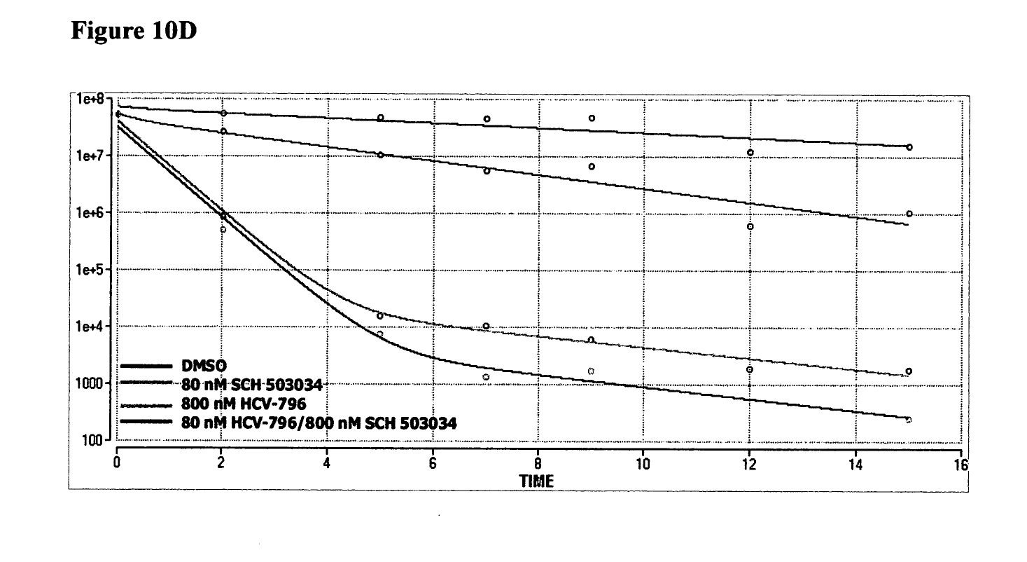

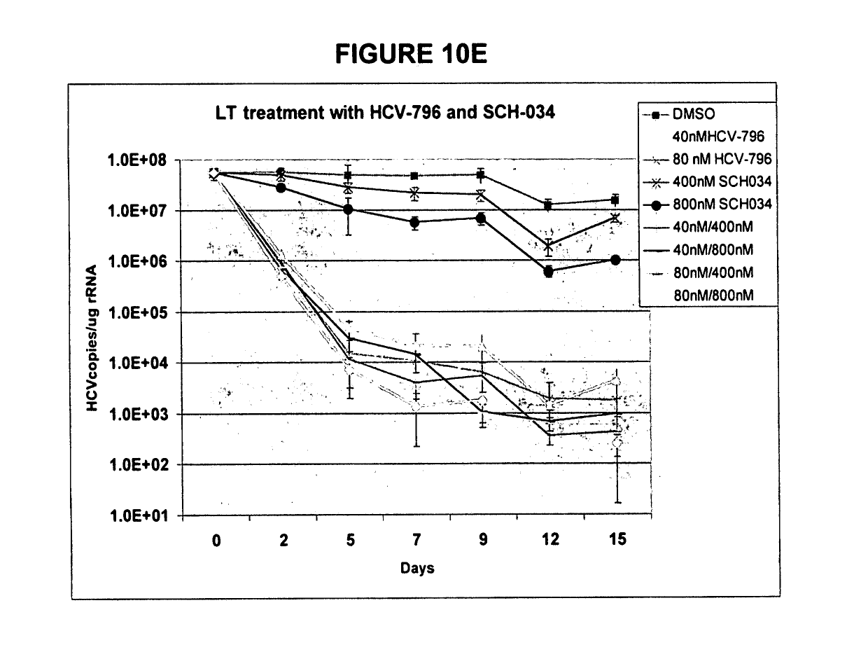

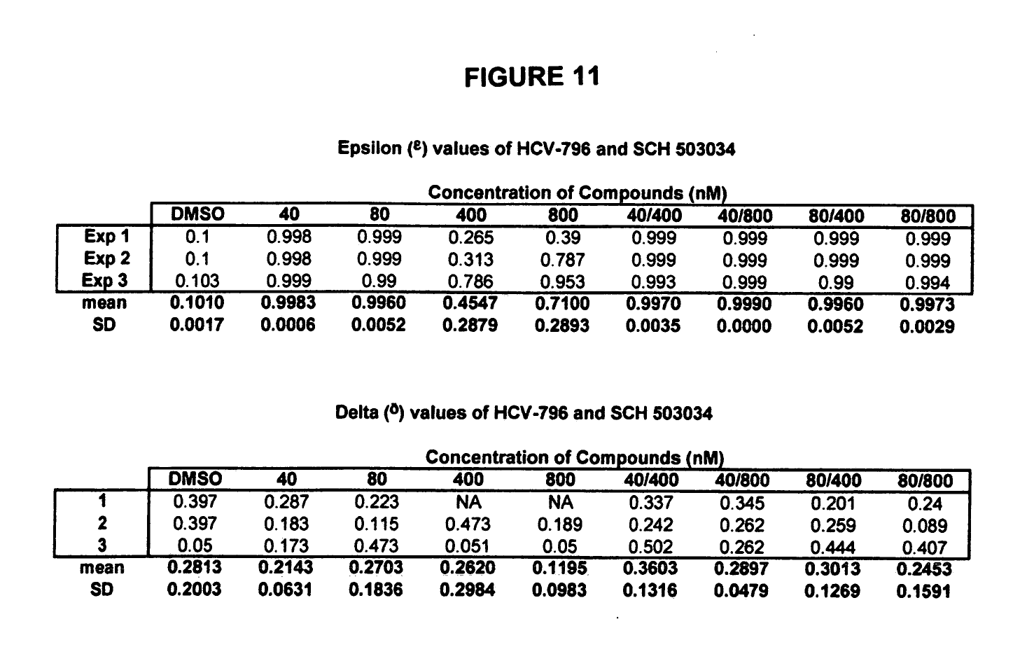

The data for HCV levels from one of three comparable studies are graphed in FIG. 10, panels A-D. The impact of combination therapy on HCV RNA level, throughout the time course, is equivalent to the sum of the impact of each drug independently (within experimental error), suggesting that the anti-replicon effect is basically additive. Likewise comparison of the efficacy parameters estimates (ε and δ, the slope of the first and second exponential phases, respectively) using the Perelson bi-exponential model (Neumann, A. U et al. (1998) Science, 282:103-107; Dahari H. et al. (2007) J. Virol., 81(2):750-760) for monitoring the impact of anti-HCV agents suggests that the two agents are not antagonistic (assuming a standard half-life for HCV RNA turnover of approximately 9 hr) (FIG. 11).

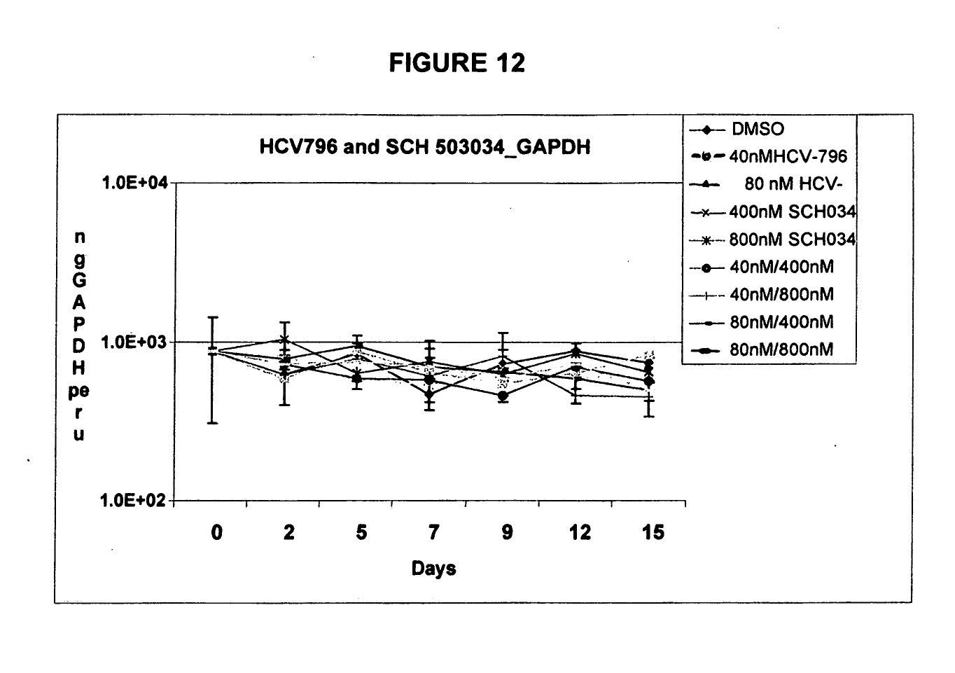

Combinations of HCV-796 and SCH 503034 do not cause any perturbation of host cell GAPDH mRNA levels (FIG. 12), suggesting that the antiviral effect is specific to HCV, and that the combination is not likely to introduce an undesirable effect on host house-keeping mRNA.

Replicon Variants with Reduced Susceptibility to HCV-796 and SCH 503034

Study A

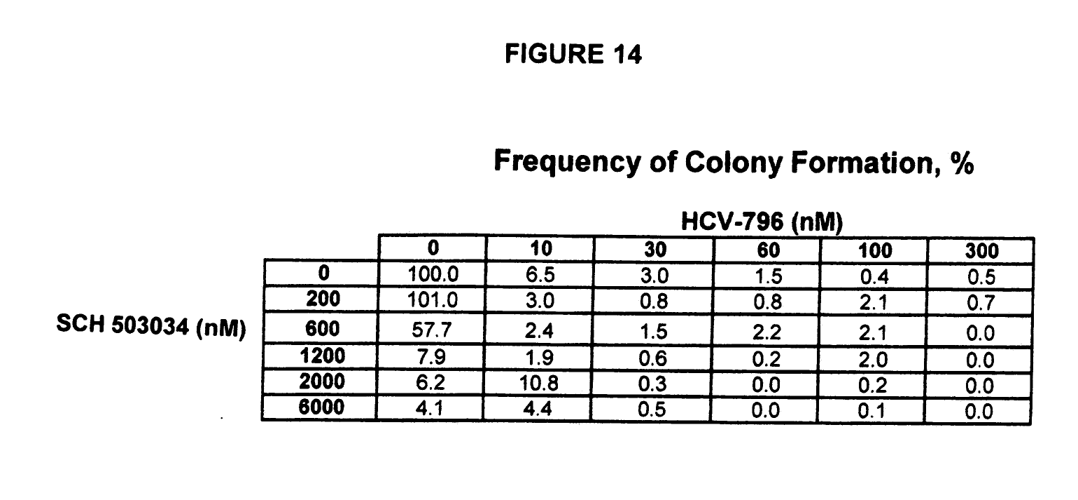

Replicon cells are treated with SCH 503034 and HCV-796, alone or in combination in the presence of G418 for 15 days (6 passages). Resistant colonies are stained with crystal violet. The number of resistant colonies is estimated by density scanning using Biorad Universal Hood II and Biorad Quantity One software for analysis. The results are shown in FIG. 14. These results indicate that the combination of SCH 503034 and HCV-796 reduces the frequency of resistant replicon formation.

Study B

Huh7-BB7 cells are plated at a density of 2-3×10⁵ cells per T25 flask and cultured in DMEM medium with 2% FCS in the absence of G418. The cells are treated with DMSO as a control, or HCV-796 alone at 40 and 80 nM, or SCH 503034 alone at 200, 400, 600 and 800 nM, or a combination of HCV-796 and SCH 503034 at 40/400, 80/400, 40/800 and 80/800 nM, respectively, for HCV-796 and SCH 503034. The DMSO concentration in both drug-treated and control cells is 0.5% (v/v). Tissue culture plates are incubated in a 37° C. incubator containing 5% CO₂. When cells reach about 80% confluence (about 2-3 days), cells are passaged in a 1:3 dilution and the old medium is replaced with fresh medium containing the compounds at the respective concentrations. Cell pellets containing 2×1 cells are collected during each passage, and monitored for HCV RNA using quantitative Taqman RT-PCR as described above. At the end of 6 passages (˜2 weeks), 0.33 mg/mL G418 is added in the presence of compounds to select for cells containing the replicon variants. During the course of selection (approximately 15-20 days), small colonies of cells resistant to the inhibitors and the antibiotic become visible. When the cell density reaches confluence, G418 at higher concentrations is added to the tissue culture medium containing inhibitors to enrich the population of replicon variants. A total of three enrichment cycles at 0.5, 0.75 and 1 mg/mL G418 are conducted to obtain the final pools of replicon variants.

Drug susceptibility of the replicon variants is evaluated as previously described in the section “Intracellular antiviral activities in HCV Replicon”. Briefly, Huh7-BB7 cells containing the replicon variants are seeded in 96-well plates at a subconfluent density in a medium containing 2% FBS without G418. SCH 503034 or HCV-796 solubilized with 100% DMSO is prepared in a 10-point, 2-fold or 3-fold dilution series, with a final DMSO concentration of 0.5% and a final volume of 200 μL. The plate is incubated for 72 hours at 37° C. and 5% CO₂ before quantification of HCV and GAPDH RNAs. The amounts of HCV, rRNA and GAPDH RNAs are quantified using the TaqMan RT-PCR assay as described above (see Quantification of HCV and 18S Ribosomal RNAs).

Prolonged treatment of replicon-containing cells with suboptimal concentrations of up to 80 nM HCV-796 alone, 800 nM SCH 503034 alone, or combinations of 40/400 and 40/800 nM HCV-796 and SCH 503034, respectively, result in selection of replicon variants that have reduced susceptibility to these compounds. No resistant replicon variants can be selected in cells treated with the 2-drug combination at concentrations of 80/400 and 80/800 nM HCV-796 and SCH 503034, respectively.

Evaluation of Effect of HCV-796/SCH 503034 Combination on Replicon RNA levels in Long Term and Short Term Assays and Emergence of Resistant Colonies in a Long Term Assay

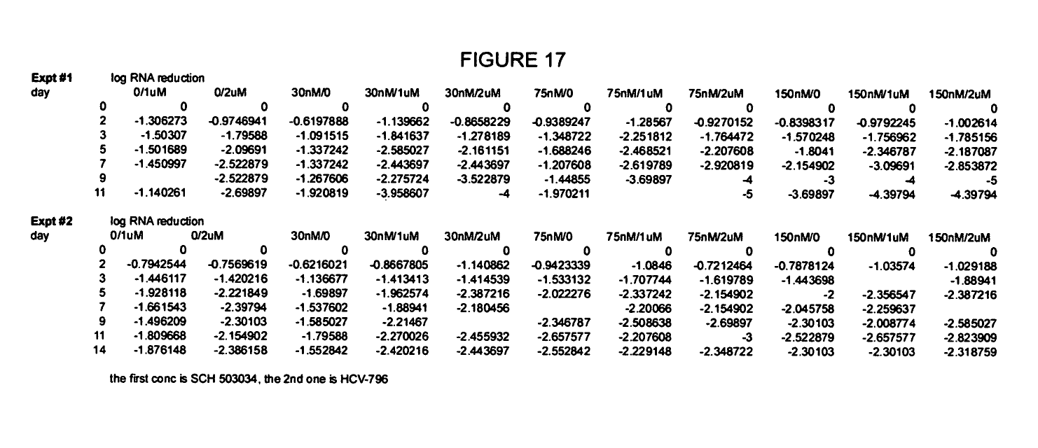

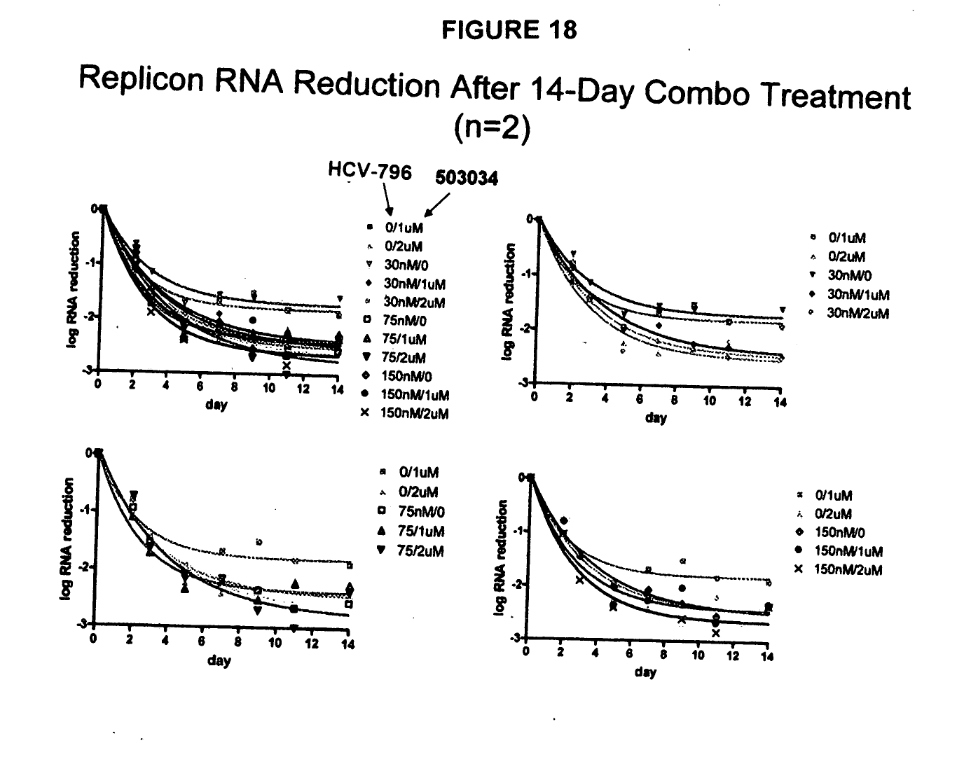

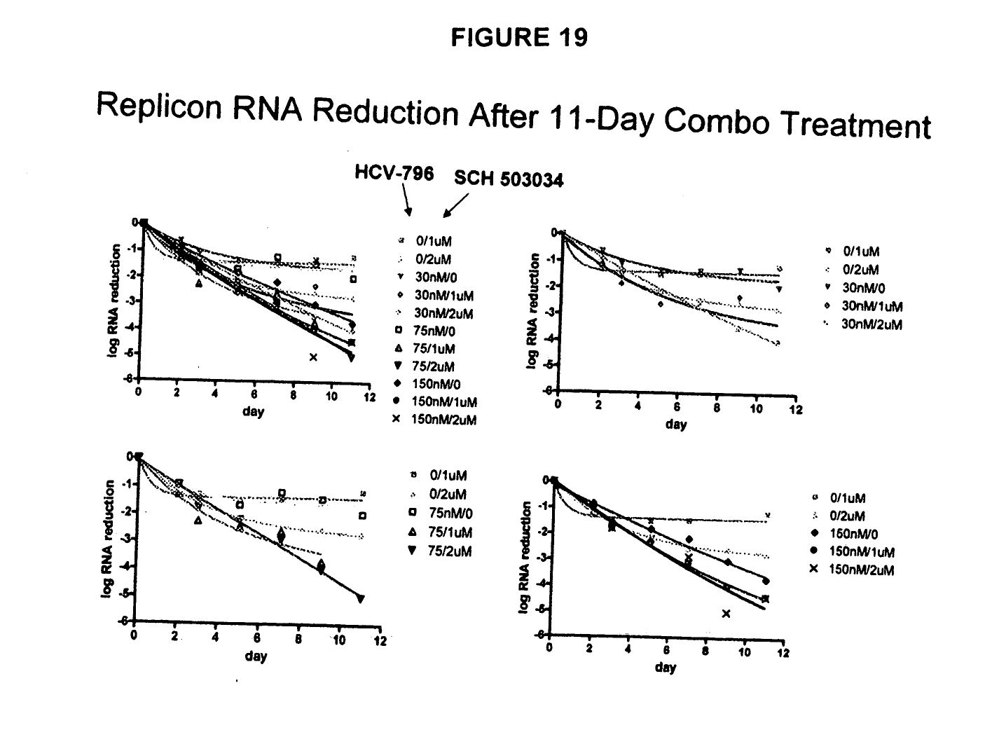

Clone 16 replicon cells are plated at a density of 8×10⁴ cells in 6-well plate and cultured in DMEM medium with 10% FCS in the absence of G418. The cells are treated with various concentrations of HCV-796 and SCH 503034 as indicated in FIGS. 17-19. All curves of FIGS. 18 and 19 are fitted using a one-phase exponential decay model; the raw data are in FIG. 17. The final DMSO concentration in both drug-treated and control cells is 1% (v/v). Tissue culture plates are incubated in a 37° C. incubator containing 5% CO₂. Compounds are refreshed every 2-3 days, and cells are passaged in a 1:3 or o 1:6 dilution (depending on the next harvest schedule) when becoming confluent. As a control, replicon cells are passaged in parallel with the same medium except no compound is added. Cells from one well of 6-well plate are collected every 2-3 days, divided into three pellets, and stored at −80° C. When all time points are harvested, one of the three cell pellets are lysed in 400 μL Ambion Cell Lysis Buffer (Cat# B8721) and heated for 5 min at 75° C. For Taqman assay, lysate is diluted 1:10 or 1:20 in water, and 4 μL of the diluted lysates are used in 384-well quantitative Taqman RT-PCR as described previously. Two independent experiments were carried out, in one experiment the cells were treated for 14 days (FIG. 18), in another experiment the were treated for 11 days (FIG. 19).

At the end of the experiment, 1 mg/ml G418 is added to the cells to recover any replicon-containing cells. When colonies appear in ˜2 weeks, the plate is stained and the number of colonies is counted (see also [0092]).

Replicon RNA reduction is calculated as below:
dCT=5BCT−gapdhCT
ddCT=dCT−dCT of no cpd control
log RNA reduction=log(½^{ddCT})

The “log RNA reduction” at day 0 is set at zero.

The results are shown in FIG. 17 and are graphically represented in FIGS. 18 and 19. It appears that time and dose-dependent inhibition of replicon RNA is observed with both inhibitors. Most notably, combination treatment achieves more significant viral RNA reduction than either single agent.

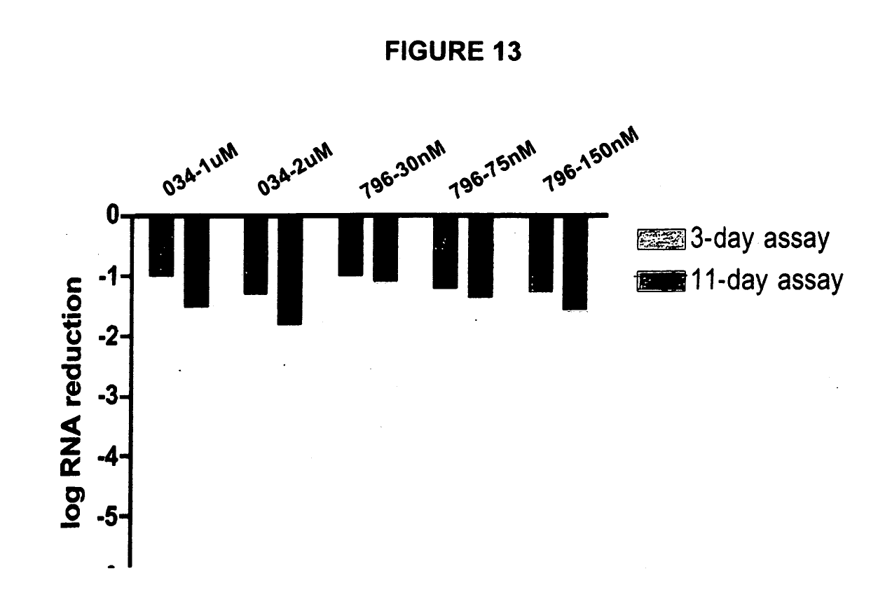

Dose responses to SCH 503034 and HCV 796 on day 3 from regular 3-day dosing and long term (11 day) dosing have been compared. The results are shown in FIG. 13. As shown, similar results are obtained from both assays.

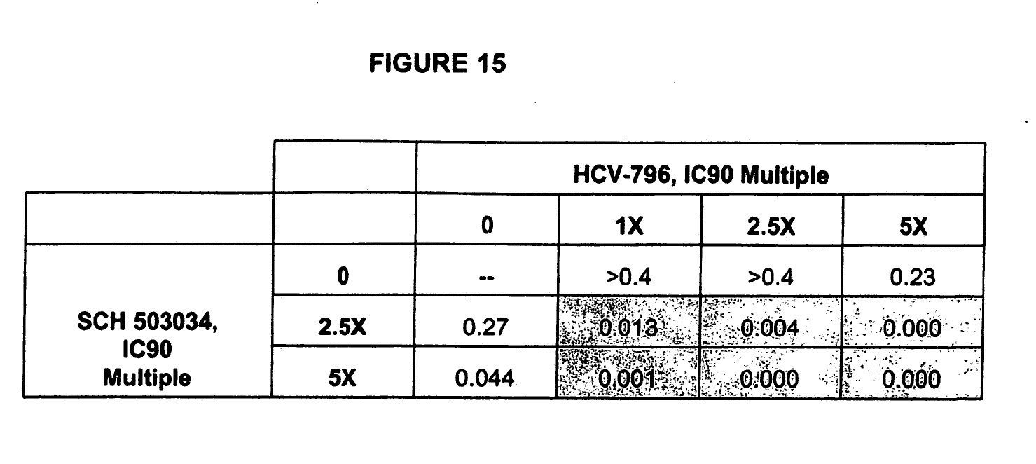

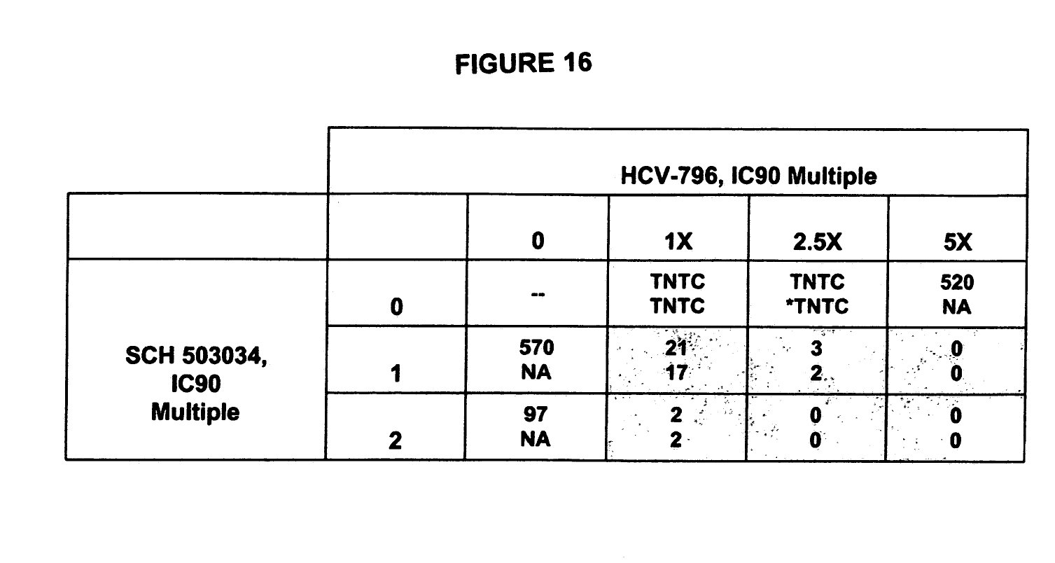

The following procedure is followed to study the frequency of emergence of resistant colonies. As above, replicon cells are treated with combinations of HCV-796 and SCH 503034 for 11-14 days in the absence of G418 selection. On the last day of treatment, G418 (1 mg/ml) selection was initiated on replicate plates for analysis of frequency of emergence of resistant colonies. The frequency of emergence of resistant colonies is also analyzed by dosing replicon cells with compounds and G418 (1 mg/ml) through the treatment period. The dosages used for both compounds are multiples of IC₉₀. As noted above, IC₉₀ is the drug dose necessary to achieve dCT=3.2 over the baseline. The results are shown in FIGS. 15 and 16. These results indicate that the frequency of emergence of resistant colonies was significantly reduced by combination treatment.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

What is claimed is:

1. A combination comprising:
(a) an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an HCV protease inhibitor, (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing.

2. The combination according to claim 1, wherein said combination further comprises a carrier, excipient and/or diluent.

3. The combination according to claim 1, wherein said combination further comprises at least one other biologically active agent.

4. The combination according to claim 3, wherein said biologically active agent is selected from the group consisting of one or more of protease inhibitors, RNA polymerase inhibitors, small interfering RNA compounds, anti-sense compounds, nucleotide analogs, nucleoside analogs, immunoglobulins, immunomodulators, hepatoprotectants, anti-inflammatory agents, antibiotics, anitvirals, and anti-infective compounds.

5. The combination according to claim 3, wherein said biologically active agent is selected from the group consisting of interferon, PEG-interferon and ribavirin.

6. The combination according to claim 3, wherein said combination further comprises at least two other biologically active agents.

7. The combination according to claim 6, wherein said biologically active agents are ribavirin and interferon or PEG-interferon.

8. The combination of claim 1 wherein said 5-cyclopropyl-2-(4-fluoro-phenyl)-6-[(2-hydroxyethyl)-methanesulfonyl-amino]-benzofuran-3-carboxylic acid methylamide is present in the form of a pharmaceutically acceptable salt.

9. The combination according to claim 8, wherein the pharmaceutically acceptable salt is selected from the group consisting of hydrochloric, sulfuric, acetic, lactic, sodium, potassium, piperidine and ammonium or a combination of two or more of the foregoing.

10. The combination according to claim 1, wherein the combination comprises an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide and an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide.

11. The combination according to claim 1, wherein the combination is a composition.

12. A method for modulating the growth of HCV in a cell in a subject in need thereof comprising administering to said subject:
(a) an amount of an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an amount of an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing,
wherein said amounts are effective to modulate growth of HCV in said cells in said subject.

13. A method for modulating the growth of HCV in one or more cells in a subject in need thereof comprising administering to said subject an amount of the composition of claim 9 effective to modulate the growth of HCV in said cells of said subject.

14. A method for treatment of disorders associated with hepatitis C virus comprising administering to a subject in need thereof:
(a) an amount of an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an amount of an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing,
wherein said amounts are effective to treat said disorders.

15. The method according to claim 14, wherein said subject is a mammal.

16. The method according to claim 14, wherein said subject is a human.

17. The method according to claim 14, wherein the HCV RNA polymerase inhibitor and the HCV protease inhibitor are administered orally, subcutaneously or parenterally.

18. The method according to claim 14, wherein the HCV RNA polymerase inhibitor and HCV protease inhibitor are administered sequentially.

19. The method according to claim 14, wherein the HCV RNA polymerase inhibitor and HCV protease inhibitor are administered concurrently.

20. The method according to claim 14, wherein the HCV RNA polymerase inhibitor and HCV protease inhibitor are administered in combination intermittently.

21. A method for treatment of disorders associated with hepatitis C virus comprising administering to a subject in need thereof an amount of the composition of claim 11 effective to treat said disorders.

22. A method of modulating HCV RNA polymerase activity and HCV protease activity in one or more HCV infected cells in a subject in need thereof comprising administering to said subject:
(a) an amount of an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an amount of an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing,
wherein said amounts are effective to treat said disorders.

23. A pharmaceutical composition for use in the treatment of disorders associated with HCV comprising:
(a) an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing.

24. A pharmaceutical composition for modulating the growth of HCV in one or more cells in a subject comprising:
(a) an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing.

25. A method of modulating HCV RNA production in one or more HCV infected cells in a subject comprising administering to said subject:
(a) an amount of an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an amount of an HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing,
wherein said amounts are effective to modulate HCV RNA production in said cells in said subject.

26. The method according to claim 25, wherein the rate of HCV RNA production is modulated.

27. A method for decreasing the emergence of resistance to an HCV polymerase inhibitor 5-cyclopropyl-2-(4-fluoro-phenyl)-6-[(2-hydroxy-ethyl)-methanesulfonyl-amino]-benzofuran-3-carboxylic acid methylamide or a HCV protease inhibitor (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide in HCV infected cells in a subject comprising administering to said subject an amount of the combination of claim 1 effective to decrease the emergence of said resistance.

28. A kit comprising:
(a) an HCV RNA polymerase inhibitor, 5-cyclopropyl-2-(4-fluoro-phenyl)-6-[(2-hydroxy-ethyl)-methanesulfonyl-amino]-benzofuran-3-carboxylic acid methylamide or a rotamer, tautomer or other isomeric form of said polymerase inhibitor or a pharmaceutically acceptable salt of any of the foregoing, and
(b) an HCV protease inhibitor, (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide or an enantiomer, stereoisomer, rotamer, tautomer, racemate or other isomeric form of said protease inhibitor or a pharmaceutically acceptable salt of any of the foregoing.
